(19) United States
(12) Patent Application Publication
(10) Pub. No.: US 2005/0112646 A1
(43) Pub. Date: May 26, 2005

(54) Unit for detecting interaction between substances utilizing projected opposed electrodes, and bioassay substrate provided with the detecting unit

(76) Inventor: Yuji Segawa, Tokyo (JP)

Correspondence Address: FROMMER LAWRENCE & HAUG LLP, 745 FIFTH AVENUE, NEW YORK, NY, 10151, US

(21) Appl. No.: 10/955,378

(22) Filed: Sep. 30, 2004

(30) Foreign Application Priority Data
Oct. 2, 2003 (JP) 2003-343955

(51) Int. Cl. C12Q 1/68; C12M 1/34

(52) U.S. Cl. 435/6; 435/4; 435/287.2; 205/777.5

(57) ABSTRACT

Disclosed are a unit for detecting an interaction between substances, a bioassay substrate including the detecting unit, and a preferable method of manufacturing the detecting unit. The detecting unit includes: a reaction region for providing sites for the interaction, such as hybridization, between the substances; and opposed electrodes disposed oppositely to each other so as to make it possible to impress an electric field on a medium, such as an aqueous solution and a gel, contained in the reaction region, wherein each of electrodes constituting the opposed electrodes have projected electrode portions projected toward the reaction region.

### BACKGROUND OF THE INVENTION

The present invention relates to a technology of arranging opposed electrodes in a reaction region for providing sites for an interaction between substances, and applying a predetermined electric field, to thereby perform a control of the high-order structures of the substances, movements of the substances, fixation of the substances, removal of unnecessary substances, etc.

A principal background art relating to the present invention will be described. First, a first background art (related art) is a technology concerning a bioassay integrated substrate so-called DNA chips or DNA microarrays (hereinafter referred to generically “DNA chips”) in which predetermined DNAs are finely arranged by the microarray technique (see, for example, Japanese Patent Laid-open No. Hei 4-505763, and WO98/503841).

The DNA chip technology uses a structure in which a multiplicity of kinds of and a multiplicity of DNA oligo-chains, cDNAs (complementary DNAs) and the like are integrated on a glass substrate or a silicon substrate, and is characterized in that it is possible to perform collective analysis of intermolecular interactions such as hybridization. Therefore, DNA chips have been utilized for analysis of variations in genes, SNPs (single nucleotide polymorphisms) analysis, gene expression frequency analysis, etc. and has come to be utilized widely in drug development, clinical diagnosis, pharmacological genomics, forensic medicine and other fields. Other than the DNA chips, there have also been developed protein chips including proteins on a substrate, biosensor chips for analyzing interactions between various substances, and the like.

A second background art is a technology concerning actions of an electric field on substances present in an electrically charged state in a liquid phase. Specifically, it is known that a nucleotide chain (nucleic acid molecule) is stretched or moved under the action of an electric field in a liquid phase. The principle of this phenomenon is considered as follows. Phosphate ions (negative charges) constituting the skeleton of the nucleotide chain and hydrogen atoms (positive charges) formed by ionization of water present in the surroundings of the phosphate ions are considered to be forming ionic fogs, the polarization vectors (dipoles) generated by the negative charges and the positive charges are as a whole aligned in one direction upon application of a high-frequency high voltage, with the result of extension of the nucleotide chain, and, in addition, when a nonuniform electric field with electric lines of force concentrated on a portion is impressed, the nucleotide chain is moved toward the portion on which the electric lines of force are concentrated (see Seiichi Suzuki, Takeshi Yamanashi, Shin-ichi Tazawa, Osamu Kurosawa and Masao Washizu: “Quantitative analysis on electrostatic orientation of DNA in stationary AC electric field using fluorescence anisotropy”, IEEE Transaction on Industrial Applications, Vol. 34, No. 1, pp.75-83 (1998)). Besides, it is known that when a DNA solution is placed in fine electrodes having a gap of several tens to several hundreds of micrometers and a high-frequency electric field of about 1 MV/m and 1 MHz is applied thereto, dielectric polarization occurs in the DNA present in a random coil form, resulting in that the DNA molecule is stretched in a straight line form in parallel to the electric field. Then, by this electrodynamic effect called “dielectricphoresis”, the polarized DNA is spontaneously drawn to the electrode end, and is fixed in the form of having one end in contact with the electrode edge (see Masao Washizu, “DNA handling conducted while viewing”, Visualized Information, Vol. 20, No. 76 (January, 2000)).

The above-mentioned DNA chip technology is a technology in which a reaction region for providing sites for an interaction between substances in a medium is preliminarily set on a substrate, and a detection nucleotide chain such as a probe DNA is preliminarily fixed in the reaction region, to thereby analyze the hybridization which is an interaction between the detection nucleotide chain and a complementary target nucleotide chain.

In the DNA chip technology, however, there have been the problems that: (1) the fixed detection nucleotide chain shows a high-order structure in which it is entangled or rounded in a random coil form under the action of Brownian motion; (2) an interference (e.g., adhesion or contact) between the fixed detection nucleotide chain and the surrounding surfaces occurs; (3) there is a deviation in the integration density of the detection nucleotide chains on the fixation surface: and (4) non-complementary nucleotide chains and surplus intercalators are present in the vicinity of the fixed detection nucleotide chain.

Hitherto, it has been impossible to solve these problems. At the time of hybridization, therefore, a steric hindrance due to the high-order structured or non-complementary nucleotide chains occurs. Accordingly, the DNA chip technology has had the technical problems that the hybridization efficiency is poor, a long time is taken to achieve the reaction, and pseudo-positivity and pseudo-negativity are generated, with the result of a lowering in detection accuracy.

### SUMMARY OF THE INVENTION

Accordingly, it is an object of the present invention to provide a detecting unit with which it is possible to freely perform a control of high-order structures of substance, movement of the substances, fixation of the substances, removal of unnecessary substances, etc., and a bioassay substrate provided with the detecting unit.

According to an aspect of the present invention, there is provided a unit for detecting an interaction between substances including:
a reaction region for providing sites for the interaction between the substances; and
opposed electrodes disposed oppositely so as to make it possible to apply an electric field to a medium contained in the reaction region;
wherein each of electrodes constituting the opposed electrodes is in the form of being projected toward the reaction region.

According to another aspect of the present invention, there is provided a bioassay substrate which includes an interaction detecting unit having:
a reaction region for providing sites for the interaction between the substances; and
opposed electrodes disposed oppositely so as to make it possible to apply an electric field to a medium contained in the reaction region;
wherein each of electrodes constituting the opposed electrodes is in the form of being projected toward the reaction region.

According to still another aspect of the present invention, there is provided a method of manufacturing an interaction detecting unit including the steps of:
forming an electrode layer composed of a predetermined projected electrode pattern on a substrate;
laminating a photosensitive resin layer on the electrode layer;
conducting dry etching with the resin layer and the electrode layer as masks; and
conducting wet etching on the lower side of the projected electrode portion to form projected electrodes.

According to the present invention, the high-order structure of a detection nucleotide such as DNA probe or a target nucleotide chain can be put from a random coil form into a stretched state under the action of an electric field applied, so that it is possible to obviate steric hindrances at the time of the interaction such as hybridization. By the action of the electric field, it is possible to align and fix the detection substance on the electrode surfaces, and to enhance the concentrations of the detection substance and the target substances on the surfaces. By these effects, the efficiency and accuracy of the interaction are enhanced, so that the operation time can be shortened, and, since the generation of pseudo-positivity or pseudo-negativity is restrained, the detection accuracy can be enhanced.

The present invention promises a high efficiency of the interaction such as hybridization at the detecting unit, so that it is possible to largely shorten the time required for the interaction. Besides, since it is possible to form an environment promising an easy progress of the interaction with high accuracy, it is possible to suppress the generation of pseudo-positivity or pseudo-negativity. Therefore, the present invention can be utilized for a bioassay substrate such as DNA chip which has such characteristics that the efficiency of the assay operation for interaction detection is excellent and that the detection accuracy is high.

While preferred embodiments of the present invention have been described using specific terms, such description is for illustrative purposes only, and it is to be understood that changes and variations may be made without departing from the spirit or scope of the following claims.

### BRIEF DESCRIPTION OF THE DRAWINGS

The above and other objects, features and advantages of the present invention will become apparent from the following description and appended claims, taken in conjunction with the accompanying drawings, in which:

FIG. 1 is a top plan view schematically showing the concept of the basic configuration of a unit for detecting an interaction between substances according to the present invention;

FIG. 2 is a top plan view schematically showing the configuration of a modified form of the detecting unit according to the present invention;

FIG. 3 is a sectional view along arrows of line I-I of FIG. 2;

FIG. 4 is a plan view of opposed electrodes viewed along arrows of line II-II of FIG. 3;

FIG. 5 is a plan view showing the form configuration of opposed electrodes in a detecting unit of a modified form;

FIG. 6 is a diagram for illustrating an example of a step concerning the interaction detection by use of the detecting unit according to the present invention (a diagram showing the manner of a DNA probe fixation step);

FIG. 7 is a diagram for illustrating an example of a step concerning the interaction detection by use of the detecting unit according to the present invention (a diagram showing the manner of a target DNA stretching and drawing step);

FIG. 8 is a diagram showing the configuration of a detecting unit provided with intersecting electrodes;

FIG. 9 is a diagram for illustrating an example of a method of manufacturing the detecting unit according to the present invention (stage of laminating an electrode layer);

FIG. 10 is a diagram for illustrating the example of the method of manufacturing the detecting unit according to the present invention (stage of laminating a resin layer);

FIG. 11 is a diagram for illustrating the example of the method of manufacturing the detecting unit according to the present invention (dry etching stage);

FIG. 12 is a diagram for illustrating the example of the method of manufacturing the detecting unit according to the present invention (soft etching stage); and

FIG. 13 is a diagram showing an example of a disk form substrate provided thereon with detecting units according to the present invention.

### DETAILED DESCRIPTION OF THE PREFERRED EMBODIMENTS

First, principal technical terms used in the present invention will be defined. The term “interaction” used in the present invention widely means chemical bondings inclusive of non-covalent bonding, covalent bonding, and hydrogen bonding and dissociation between substances, and includes hybridization which is a complementary bonding between nucleic acids (nucleotide chains), for example.

Next, the term “opposed electrodes” means at least one pair of electrodes which are arranged oppositely to each other.

The term “nucleotide chain” means a polymer of a phosphoric acid ester of a nucleoside in which a purine or pyrimidine base and a sugar are bonded by glycoside bonding, and widely includes oligonucleotides inclusive of probe DNAs, polynucleotides, DNAs (whole length or sections thereof) formed by polymerization of purine nucleotide with pyrimidine nucleotide, cDNAs (c probe DNAs) obtained by reverse transcription, RNAs, polyamide nucleotide derivatives (PNAs), etc.

The term “hybridization” means a complementary chain (double chain) forming reaction between nucleotide chains having complementary base sequence structures. The term “mishybridization” means the complementary chain forming reaction which is not normal.

The term “reaction region” means a region which can provide reaction sites for hybridization or other interactions, and examples thereof include a reaction site in the shape of a well capable of preserving or holding a medium such as a liquid phase and a gel. The interaction conducted in the reaction region is not narrowly limited, provided that the interaction conforms to the object or effects of the present invention. Examples of the interaction include not only an interaction between a single-chain nucleic acids, i.e., hybridization but also an interaction between peptide (or protein) and a desired double-chain nucleic acid formed from a detection nucleic acid, an enzyme response reaction and other intermolecular interactions. Where the double-chain nucleic acid is used, for example, the bonding between a receptor molecule of a hormone receptor or the like which is a transcription factor and a response sequence DNA portion, and the like can be analyzed.

The term “detection substance” is a substance which is preliminarily added into the reaction region and which is present in a free state in the region, or a substance which is present in the state of being fixed to a predetermined surface portion of the reaction region. The detection substance is a substance for capturing and detecting a substance showing a specific interaction with the substance, and includes detection nucleotide chains such as DNA probes.

The term “target substance” means a substance which serves as a target of an interaction with the detection substance, and examples thereof include a nucleotide chain having a base sequence complementary to the DNA probe.

The term “steric hindrance” means a phenomenon in which due to the presence of a bulky substituent group in the vicinity of a reaction center or the like in a molecule, the posture of a reaction molecule, or the steric structure (high-order structure), the access of molecules of the medium species becomes difficult and, as a result, it becomes difficult for the desired reaction (hybridization, in the present patent application) to take place.

The term “dielectricphoresis” is a phenomenon in which molecules are driven toward the higher electric field side in a field where the electric field is anisotropic. Further, where an AC voltage is applied, the polarity of polarization is reversed attendant on the reversion of the polarity of the applied voltage, so that the driving effect can be obtained in the same manner as in the case of DC (see “Micromachines and Material Technology (published by CMC Publishing Co., Ltd.)” complied under the supervision of Teru Hayashi, pp.37-46, Chapter 5, Cell and DNA manipulation).

The term “bioassay substrate” means an information integration substrate used for the purpose of biochemical or molecular biological analysis, and includes the so-called DNA chip.

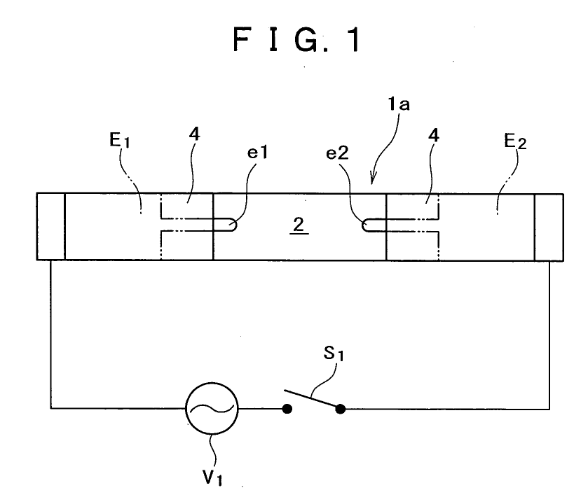

Now, a preferred embodiment of the present invention will be described below referring to the accompanying drawings. First, FIG. 1 is a top plan view schematically showing the concept of the basic configuration of a unit for detecting an interaction between substances (hereinafter referred to simply as “detecting unit”) according to the present invention.

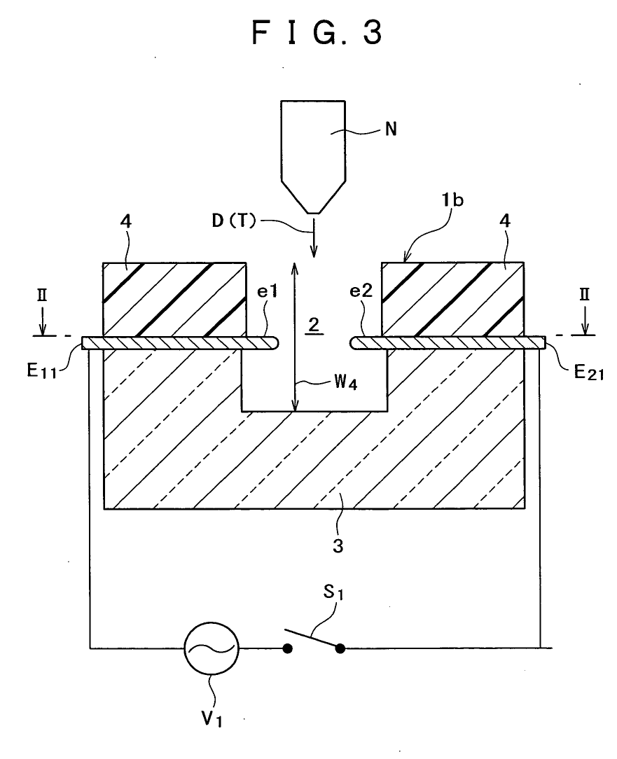

Symbol 1a in FIG. 1 denotes an essential part of the most basic embodiment of the detecting unit according to the present invention. The detecting unit 1a is formed on a substrate (see symbol 3 in FIG. 3 and the like) formed, for example, of a glass, synthetic resin or the like, and is a portion devised for detecting an interaction between substances.

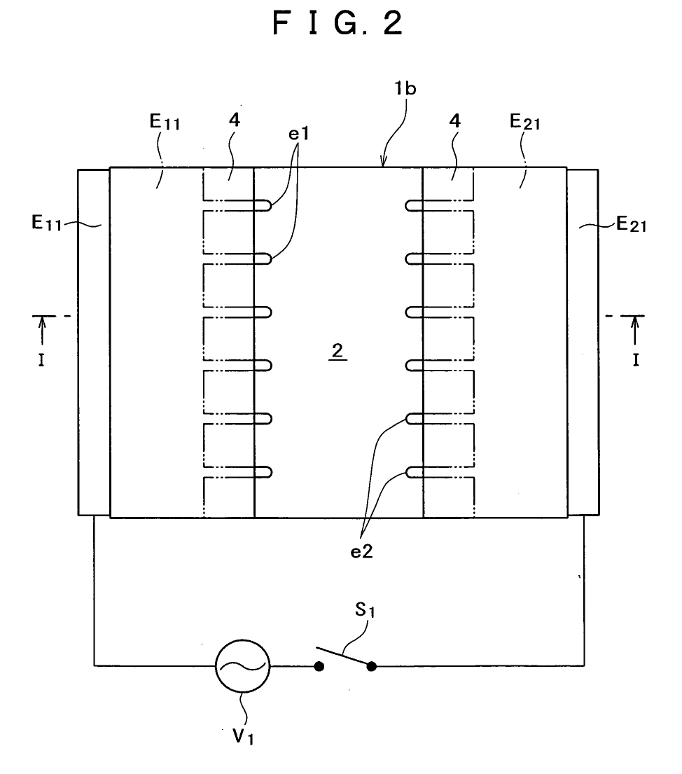

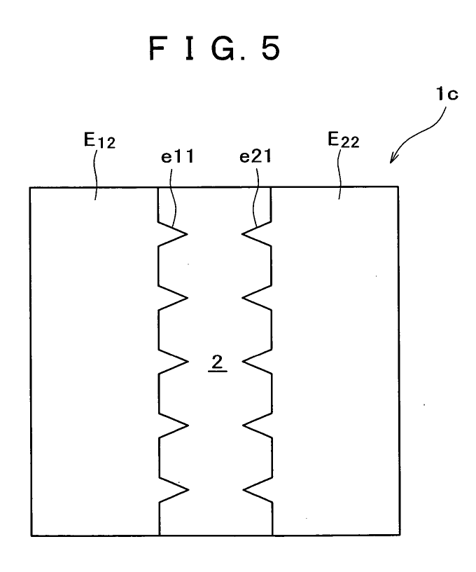

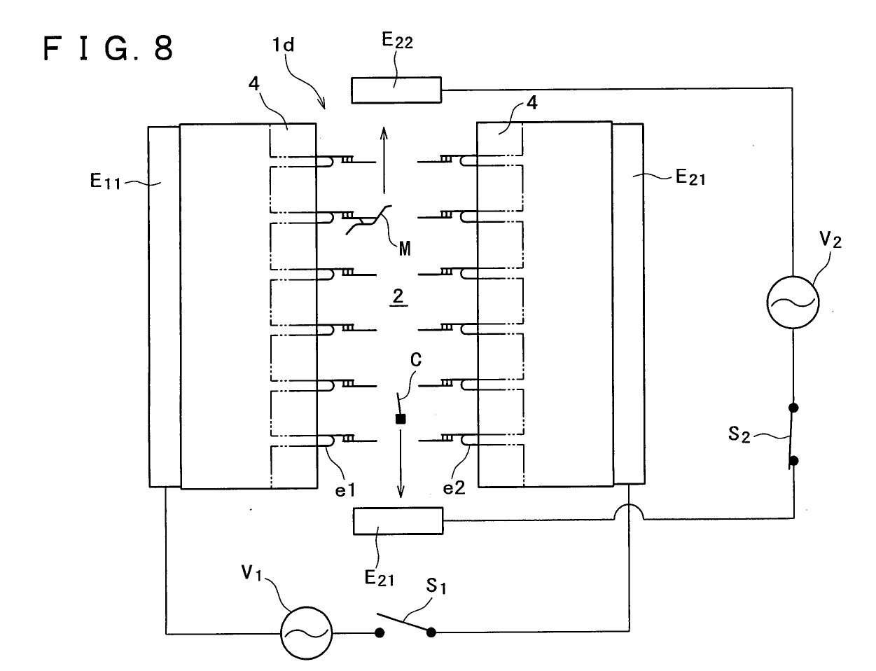

The detecting unit la and other detecting units 1b (FIG. 2), 1c (FIG. 5), and 1d (FIG. 8) are each provided with a reaction region 2 having a predetermined volume capable of preserving or holding a medium such as an aqueous solution and a gel which serves as sites for the interaction between the substances, and a pair of opposed electrodes E₁, E₂ disposed oppositely to each other on both sides of the reaction region 2.

The opposed electrodes E₁, E₂ can be formed of a metal such as gold and aluminum or of a conductor other than metal; for example, they can be formed of a transparent conductor such as ITO (Indium Tin Oxide). Incidentally, the opposed electrodes E₁, E₂ are connected to a power source V₁ shown, by turning ON a switch S₁.

The opposed electrodes E₁, E₂ are each formed in the shape of being projected toward the reaction region 2, and include projected electrode portions e1, e2 in a needle-like or rod-like form which are opposed to each other.

Of the opposed electrodes E₁, E₂, each surface on the side for fronting the reaction region 2 is covered with an insulation layer (not shown). The insulation layer plays the role of preventing an electrochemical reaction due to an ionic solution which may be preserved in the reaction region 2. The insulation layer can be formed of such a material as SiO₂, SiN, SiOC, SiOF, SiC, TiO₂, etc.

FIG. 2 is a top plan view schematically showing the configuration of a modified form of the detecting unit according to the present invention. The detecting unit 1b representing a modified form includes opposed electrodes E₁₁, E₂₁ having a configuration in which the above-mentioned opposed electrodes E₁, E₂ are respectively arrayed at a predetermined regular interval. Therefore, a plurality of pairs (six pairs in the figure) of projected electrode portions e1, e2 are disposed oppositely to each other in the reaction region 2 of the detecting unit 1b.

Incidentally, though not shown in the figure, the projected electrode portions e1 constituting the electrode E₁₁ and the projected electrode portions e2 constituting the electrode E₂₁ may not necessarily be arrayed at the regular interval, and the interval can be appropriately selected. Besides, there can be adopted a configuration in which the number of the projected electrode portions on one side is greater than the number of the projected electrode portions on the other side, or a configuration in which the number density per unit length of the projected electrode portions on one side is higher than the number density per unit length of the projected electrode portions on the other side. It is considered that the electric lines of force are concentrated more on the side of the projected electrode portions with a higher number density.

Next, FIG. 3 is a sectional view along arrows of line I-I of FIG. 2. As shown in FIG. 3, the opposed electrodes E₁₁, E₂₁ are provided in close contact with the substrate 3 formed of a glass, synthetic resin or the like. On the opposed electrodes E₁₁, E₂₁ is formed an inorganic material such as SiO₂ or a synthetic resin layer 4 of a polyimide resin or the like as shown in the figure.

The reaction region 2 can be observed as a recessed portion opened to the upper side, as shown in FIG. 3. Into the reaction region 2, an aqueous solution or the like containing a detection substance D such as a DNA probe and a target substance T showing an interaction with the detection substance D is dropped from a nozzle N or the like disposed on the upper side, as shown schematically in the figure.

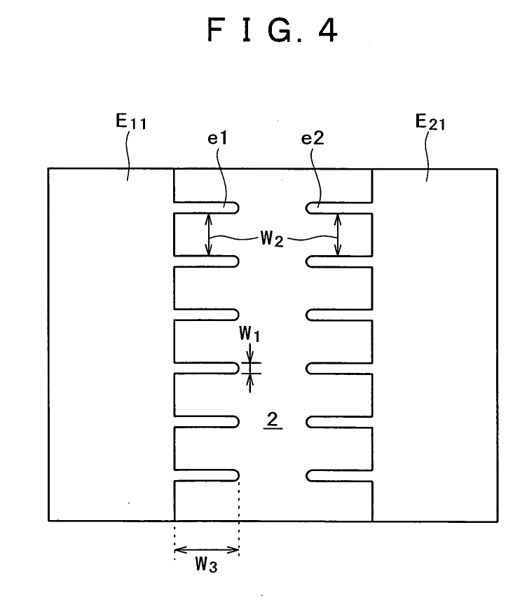

FIG. 4 is a plan view of the opposed electrodes E₁₁, E₂₁ as viewed along arrows of line II-II of FIG. 3. The width (or thickness) W₁ of the projected electrode portions e1, e2 may be set to, for example, about 0.5 μm, and the interval W₂ between the projected electrode portions e1 and e1 (or e2 and e2) may be set to, for example, about 1 to 10 μm. In addition to W₁ and W₂, the length W₃ of the projected electrode portions e1, e2 and the depth W₄ (see FIG. 3) of the reaction region 2 may be appropriately determined according to the molecular lengths of the detection substance D and the target substance T to be dealt with.

FIG. 5 is a plan view showing the form configuration of opposed electrodes E₁₂, E₂₂ in a detecting unit 1c representing another modified embodiment. The opposed electrodes E₁₂, E₂₂ have projected electrode portions e11, e21 which are pointed in triangular shape. Thus, the projected electrode portions according to the present invention may be appropriately formed in any shape that has such an edge shape that the electric lines of force (described later) are easily concentrated thereon.

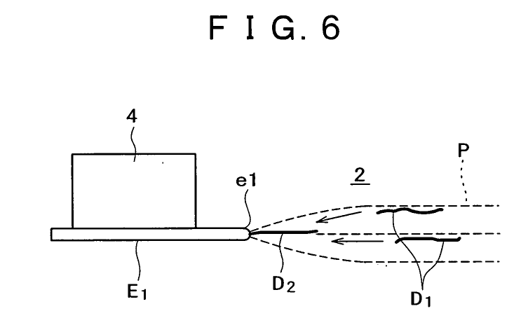

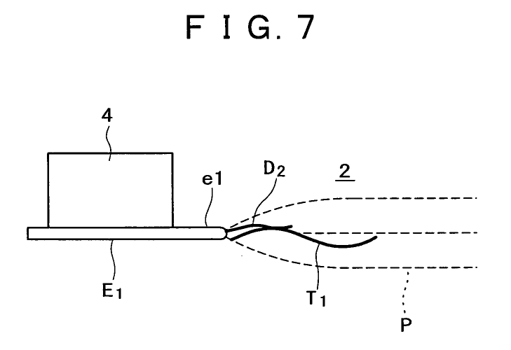

Now, referring to FIGS. 6 and 7, an example of a step concerning the interaction detection by use of the detecting unit according to the present invention will be described, taking as a representative example the action at the electrode E₁ of the detecting unit la shown in FIG. 1. Incidentally, while hybridization is taken as the interaction in the step example, the interaction is not limited to the hybridization.

First, an aqueous solution containing a DNA probe D₁ as a representative example of the detection substance D is dropped in a predetermined quantity from a nozzle (see FIG. 3) into a reaction region 2. Next, a switch S₁ is turned ON, to impress an AC electric field from a power source V₁. As a preferable condition of the impressed electric field here, for example, about 1×10⁶ V/m and about 1 MHz can be selected preferably (see Masao Washizu and Osamu Kurosawa: “Electrostatic Manipulation of DNA in Microfabricated Structures”, IEEE Transaction on Industrial Application, Vol. 26, No. 26, pp.1165-1172 (1990)). Incidentally, the DNA probe D₁ at the time of being dropped has a random coil form high-order structure under the action of the Brownian motion.

By the application of the electric field, the DNA probe D₁ in the librated state denoted by symbol D₁ in FIG. 6 is moved by dielectricphoresis toward the projected electrode portion e1 while being stretched along the AC electric field, and, finally, its terminal end portion is fixed to the projected electrode portion e1 where the electric lines of force P are concentrated. Incidentally, symbol D₂ in FIG. 6 denotes the fixed DNA probe.

Incidentally, in the case where the surface of the projected electrode portion e1 is surface treated with streptoavidin, the system is suitable for fixation of the terminal end of the viotinated DNA probe. Alternatively, in the case where the surface of the projected electrode portion e1 is surface treated with a thiol (SH) group, the system is suitable for fixing the DNA probe, modified with the thiol group at the terminal end thereof, by a disulfide bond (—S—S-bond).

After the fixation of the DNA probe D₁ by the above-mentioned method is finished, the assembly is washed with a predetermined buffer solution (e.g., phosphate buffered saline), whereby surplus DNA probes and the DNA probes non-specifically adsorbed on the surface of the projected electrode portion e1 can be removed from the reaction region 2.

Subsequently, a solution containing a target DNA as a representative example of the target substance T shown in FIG. 3 is dropped into the reaction region 2, and thereafter the switch S₁ shown in FIG. 1 and the like is turned ON, to impress an AC electric field. As the electric field condition in this case, also, for example, about 1×10⁶ V/m and about 1 MHz can be preferably selected (see Masao Washizu and Osamu Kurosawa: “Electrostatic Manipulation of DNA in Microfabricated Structures”, IEEE Transaction on Industrial Application, Vol. 26, No. 26, pp.1165-1172 (1990)).

Upon the application of the electric field, the target DNA denoted by symbol T₁ in FIG. 7 is also moved by dielectricphoresis toward the projected electrode portion e1 while being stretched along the AC electric field, and, finally, it is moved to the vicinity of the projected electrode portion e1 where the electric lines of force P are concentrated. Incidentally, at the time of dropping the target DNA-containing solution into the reaction region 2, an intercalator capable of being selectively inserted and bonded to a double chain portion may be dropped simultaneously.

Next, the switch S₁ is turned OFF (see FIG. 1), to stop the application of the AC electric field, and the hybridization is made to proceed under the natural Brownian motion. FIG. 7 schematically shows the condition where the hybridization has proceeded between the fixed DNA probe D₂ and the target DNA in the stretched state denoted by symbol T₁. Incidentally, the intercalator may be dropped into the reaction region 2 after the hybridization.

Generally, the target DNA denoted by symbol T (T₁) is longer than the DNA probe D₂; therefore, the target DNAs may interfere with each other in the narrow reaction region 2 to bring about a steric hindrance, which hampers the hybridization, or they may adhere to wall surfaces of the reaction region 2 in the vicinity of the fixation surface. Thus, the progress of the hybridization may often be inhibited.

In the present invention, on the other hand, the projected electrode portions e1 and e2 forming a nonuniform electric field have electrode edges present at positions far from the surrounding wall surfaces, and the projected electrode portions can be spaced from each other (see FIGS. 4 and 5). Therefore, there is secured a sufficient space for hybridization, so that a steric hindrance is generated with difficulty.

Incidentally, as shown in the modified embodiment in FIG. 8, there may be disposed another pair of opposed electrodes E₂₁-E₂₂ having an opposition axis intersecting (shown in the figure) vertically or horizontally to the opposed electrodes E₁₁-E₂₁. After the hybridization, a switch S₂ shown in FIG. 8 is turned ON, to impress an AC electric field on the opposed electrodes E₂₁-E₂₂ from a power source V₂, whereby the mishybridized DNA (denoted by symbol M) and the surplus intercalator C can be drawn to the opposed electrode E₂₁ or E₂₂ and removed from the detection portion.

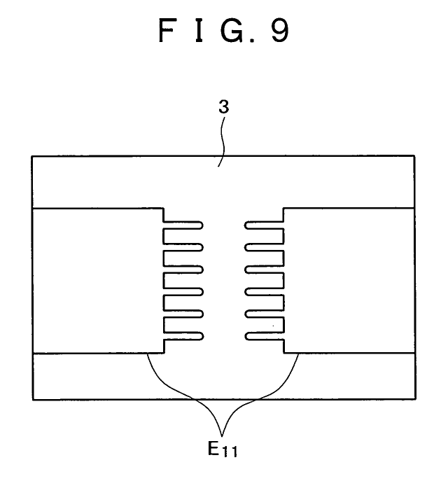

Next, based on FIGS. 9 to 12, one example of the method of manufacturing the detecting unit according to the present invention will be described. The detecting unit will be described by taking an embodiment denoted by symbol 1b as a representative example. Taking as an example the case where a substrate 3 is made of a glass, predetermined electrode layers E, E are formed on the glass substrate 3 by use of gold (see FIG. 9). In this case, for securing close contact between the glass substrate 3 and the gold-made electrode layers E, E, it is desirable to preliminarily provide a layer (not shown) of Cr, Ti or the like between the glass substrate 3 and the electrode layers E, E.

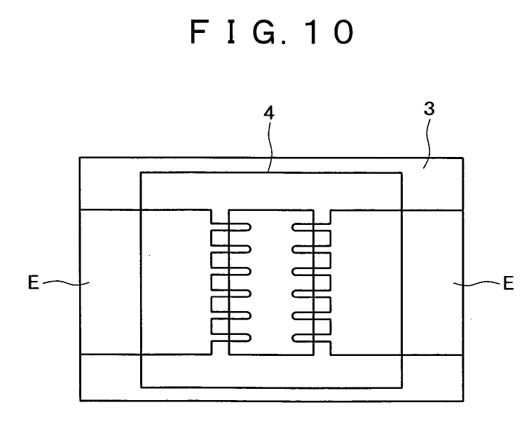

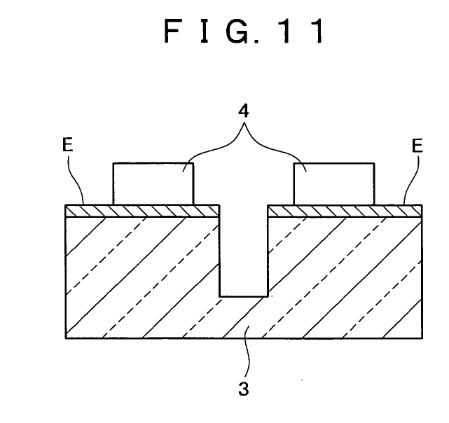

Next, as shown in FIG. 10, for example, a photosensitive resin layer (e.g., a polyimide resin layer) 4 is laminated on the electrode layers E, E, to secure the depth required of a reaction region 2. Subsequently, as shown in FIG. 11, with the resin layer 4 and the electrode layers E, E as masks, the substrate 3 is etched by dry etching technique such as RIE.

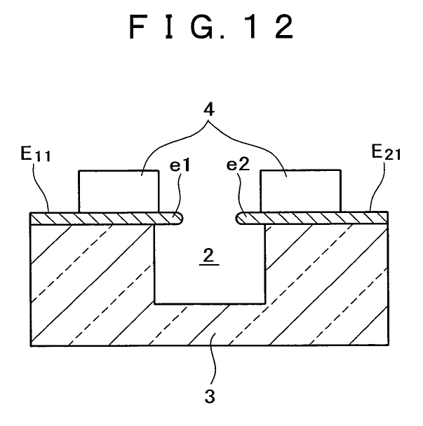

Thereafter, by use of an HF solution or the like, the lower side of projected electrode portions is wet etched, to form projected electrode portions E₁₁, E₂₁. Incidentally, since gold is not damaged by the HF solution, a projected electrode structure as shown in FIG. 12 can finally be formed.

Incidentally, the etching of the glass-made substrate 3 may be carried out at a stroke by soft etching using the HF solution, without adopting the above-mentioned dry etching technique. In the fabrication of the detecting unit according to the present invention, however, it is desirable to jointly use the dry etching and the soft etching, for enhancing controllability of the electrode shape. Besides, in the case of covering the thus formed projected electrode portions with an insulation layer, it is desirable to form a film of SiO₂ or the like by CVD, for example.

By preliminarily arranging the detecting units denoted by symbols 1a to 1d above-mentioned in a predetermined array on the substrate, a bioassay substrate such as DNA chip can be provided with which interactions such as hybridization can be made to proceed in a short time and collective analysis can be performed.

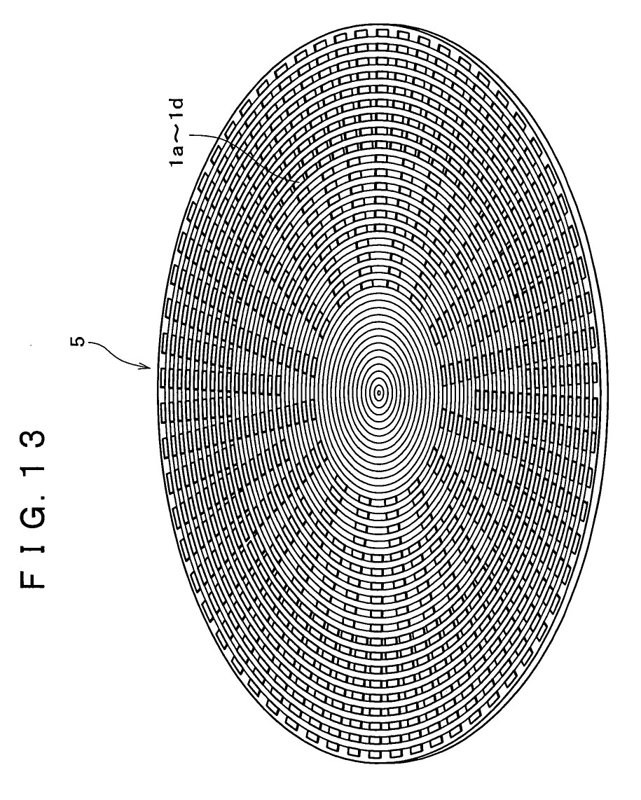

FIG. 13 is a diagram showing one example of the bioassay substrate. As shown in FIG. 13, for example, a multiplicity of detecting units 1a and the like can be arranged on a disk-like substrate 5 in such a manner that they can be divided into groups.

Incidentally, the detection of the interaction proceeding at any detecting unit 1a or the like provided on the substrate 5 can be carried out by use of a known optical detection means by which a fluorescent substance preliminarily marked onto the detection substance D fixed to the electrode surface or a fluorescent intercalator inserted and bonded to a substance (double chain nucleic acid) showing an interaction is irradiated with fluorescence exciting rays at a predetermined wavelength and the fluorescence is detected. Alternatively, a method may be adopted in which the light-emitting image of the detecting unit 1a and the like is picked up, and the quantity of light obtained from the image is quantitatively analyzed and detected.

The present invention is not limited to the details of the above described preferred embodiments. The scope of the invention is defined by the appended claims and all changes and modifications as fall within the equivalence of the scope of the claims are therefore to be embraced by the invention.

1. A unit for detecting an interaction between substances comprising:
a reaction region for providing sites for said interaction between said substances; and
opposed electrodes disposed oppositely so as to make it possible to apply an electric field to a medium contained in said reaction region;
wherein each of electrodes constituting said opposed electrodes is in the form of being projected toward said reaction region.

2. The unit for detecting an interaction between substances as set forth in claim 1, wherein said opposed electrodes are arrayed at a predetermined interval.

3. The unit for detecting an interaction between substances as set forth in claim 1, wherein an alternating current field is applied to said opposed electrodes.

4. The unit for detecting an interaction between substances as set forth in claim 3, wherein tip end portions of said opposed electrodes serve as portions for fixing a nucleotide chain for detection.

5. The unit for detecting an interaction between substances as set forth in claim 1, wherein said interaction is hybridization.

6. A bioassay substrate which comprises an interaction detecting unit including:
a reaction region for providing sites for said interaction between said substances; and
opposed electrodes disposed oppositely so as to make it possible to apply an electric field to a medium contained in said reaction region;
wherein each of electrodes constituting said opposed electrodes is in the form of being projected toward said reaction region.

7. A method of manufacturing an interaction detecting unit comprising the steps of:
forming an electrode layer composed of a predetermined projected electrode pattern on a substrate;
laminating a photosensitive resin layer on said electrode layer;
conducting dry etching with said resin layer and said electrode layer as masks; and
conducting wet etching on the lower side of said projected electrode portion to form projected electrodes.
